SEQUENCE LISTING (15 sequences)

SEQ ID NO: 1 - DNA, 18 bp, Artificial
  Synthetic nucleic acid ligand
aggaagatgtgcctttca

SEQ ID NO: 2 - DNA, 19 bp, Artificial
  Synthetic Nucleic Acid Ligand
aaatgcttgctagaccaat

SEQ ID NO: 3 - DNA, 25 bp, Artificial
  Synthetic Nucleic Acid Ligand
  misc_feature (11)..(11): C at position 11 is derivatized with dye LC Red 640
ccaccttctccaagaactatattgt

SEQ ID NO: 4 - DNA, 22 bp, Artificial
  Synthetic Nucleic Acid Ligand
cgttgacctccactcagtgtga

SEQ ID NO: 5 - DNA, 17 bp, Artificial
  Synthetic Nucleic Acid Ligand
  misc_feature (5)..(5): C at position 5 is a locked nucleic acid
  misc_feature (11)..(11): C at position 11 is derivatized with dye LC Red 640
  misc_feature (11)..(11): C at position 11 is a locked nucleic acid
  misc_feature (15)..(15): A at position 15 is a locked nucleic acid
ccaccttctccaagaac

SEQ ID NO: 6 - DNA, 17 bp, Artificial
  Synthetic Nucleic Acid Ligand
  misc_feature (4)..(4): T at position 4 is a locked nucleic acid
  misc_feature (8)..(8): C at position 8 is a locked nucleic acid
  misc_feature (13)..(13): T at position 13 is a locked nucleic acid
acctccactcagtgtga

SEQ ID NO: 7 - DNA, 17 bp, Artificial
  Synthetic Nucleic Acid Ligand
  misc_feature (10)..(11): C at position 10 and 11 is a locked nucleic acid
  misc_feature (11)..(11): C at position 11 is derivatized with dye LC Red 640
  misc_feature (12)..(12): A at position 12 is a locked nucleic acid
ccaccttctccaagaac

SEQ ID NO: 8 - DNA, 16 bp, Artificial
  Synthetic Nucleic Acid Ligand
  misc_feature (7)..(9): C at positions 7 and 9 is a locked nucleic acid
  misc_feature (8)..(8): T at position 8 is a locked nucleic acid
cctccactcagtgtga

SEQ ID NO: 9 - DNA, 15 bp, Artificial
  Synthetic Nucleic Acid Ligand
  misc_feature (5)..(5): T at position 5 is a locked nucleic acid
  misc_feature (6)..(7): C at positions 6 and 7 is a locked nucleic acid
  misc_feature (7)..(7): C at position 7 is derivatized with dye LC Red 640
  misc_feature (11)..(11): A at position 11 is a locked nucleic acid
cttctccaagaacta

SEQ ID NO: 10 - DNA, 16 bp, Artificial
  Synthetic Nucleic Acid Ligand
  misc_feature (1)..(1): C at position 1 is a locked nucleic acid
  misc_feature (4)..(4): T at position 4 is a locked nucleic acid
  misc_feature (7)..(7): G at position 7 is a locked nucleic acid
  misc_feature (11)..(11): G at position 11 is a locked nucleic acid
cactcagtgtgattcc

SEQ ID NO: 11 - DNA, 13 bp, Artificial
  Synthetic Nucleic Acid Ligand
  misc_feature (1)..(1): C at position 1 is a locked nucleic acid
  misc_feature (2)..(2): T at position 2 is a locked nucleic acid
  misc_feature (6)..(7): C at positions 6 and 7 is a locked nucleic acid
  misc_feature (7)..(7): C at position 7 is derivatized with dye LC Red 640
  misc_feature (10)..(10): G at position 10 is a locked nucleic acid
cttctccaagaac

SEQ ID NO: 12 - DNA, 14 bp, Artificial
  Synthetic Nucleic Acid Ligand
  misc_feature (1)..(1): C at position 1 is a locked nucleic acid
  misc_feature (6)..(6): T at position 6 is a locked nucleic acid
  misc_feature (7)..(7): G at position 7 is a locked nucleic acid
  misc_feature (10)..(10): A at position 10 is a locked nucleic acid
  misc_feature (13)..(13): C at position 13 is a locked nucleic acid
ctcagtgtgattcc

SEQ ID NO: 13 - DNA, 13 bp, Artificial
  Synthetic Nucleic Acid Ligand
  misc_feature (1)..(1): C at position 1 is a locked nucleic acid
  misc_feature (7)..(7): C at position 7 is derivatized with dye LC Red 640
cttctccaagaac

SEQ ID NO: 14 - DNA, 14 bp, Artificial
  Synthetic Nucleic Acid Ligand
ctcagtgtgattcc

SEQ ID NO: 15 - DNA, 13 bp, Artificial
  Synthetic Nucleic Acid Ligand
  misc_feature (7)..(7): C at position 7 is derivatized with dye LC Red 640
cttctccaagaac

(19) United States
(12) Patent Application Publication
(10) Pub. No.: US 2007/0269803 A1
(43) Pub. Date: Nov. 22, 2007

(54) Fluorogenic Nucleic Acid Probes Including Lna for Methods to Detect and/or Quantify Nucleic Acid Analytes

(76) Inventor: Khalil Arar, Meudon (FR)

Correspondence Address: SIGMA-ALDRICH CO., 3050 SPRUCE STREET, ST. LOUIS, MO, 63013, US

(21) Appl. No.: 10/560,987

(22) PCT Filed: Jun. 18, 2004
(86) PCT No.: PCT/US04/19671

§ 371 Date: Apr. 6, 2007

Related U.S. Application Data
(60) Provisional application No. 60/482,684, filed on Jun. 26, 2003.

(51) Int. Cl. C12Q 1/68; C07H 21/00

(52) U.S. Cl. 435/6; 536/24.3

(57) ABSTRACT

The present invention relates to novel methods for detecting or quantifying nucleic acid analytes through their interactions with a nucleic acid probe or a pair of nucleic acid probes, wherein the probe or the pair of probes is comprised of at least one monomeric LNA moiety and two or more dyes, wherein at least one of said dyes is fluorescent. Preferably the probe or the pair of probes is comprised of a combination of two dyes, wherein either both are fluorescent dyes that coactively function as the donor dye and the acceptor dye of a FRET system, or wherein one of said dyes is a fluorescent dye and the other is a corresponding non-fluorescent quencher dye. Included in the present invention are novel nucleic acid probes for use in the detection and quantification of analytes according to the methods of this invention. The novel nucleic acid probes of the invention are comprised of an n-meric nucleic acid comprising any number of 1 to n monomeric locked nucleic acid (LNA) moieties that may be located in any position(s) of the nucleic acid sequence. The nucleic acid probes are further characterized in that they are derivatized with one or more dyes, wherein said dyes are independently selected from fluorescent dyes or non-fluorescent quencher dyes. The methods provided by the invention are based on the change of fluorescence resulting from the hybridization of the inventive nucleic acid probes or pairs of nucleic acid probes with nucleic acid analytes.

### FIELD OF INVENTION

The present invention relates to the field of molecular biology. More specifically, the present invention relates to the field of assays that utilize nucleic acid probes to detect and/or quantify nucleic acid analytes. The subject invention will be useful in any application where it is desired to detect or quantify a nucleic acid analyte.

### BACKGROUND OF THE INVENTION

Advances in DNA technology and sequencing, specifically the sequencing of whole genomes including the human genome, have resulted in a significantly increased need to detect and/or quantify specific nucleic acid sequences. Applications include the fields of in vitro diagnostics, including clinical diagnostics, research in the fields of molecular biology, high throughput drug screening, veterinary diagnostics, agricultural-genetics testing, environmental testing, food testing, industrial process monitoring and insurance testing. In vitro diagnostics and clinical diagnostics is related to the analysis of nucleic acid samples drawn from the body to detect the existence of a disease or condition, its stage of development and/or severity, and the patient's response to treatment. In high throughput drug screening and development, nucleic acids are used similarly to other agents, such as, antigens, antibodies, receptors, etc, to analyze the response of biological systems upon exposure to libraries of compounds in a high sample number setting to identify drug leads. Veterinary diagnostics and agricultural genetics testing involve samples from a non-human animal or a plant species similar to in vitro diagnostics and to provide means of quality control for agricultural genetic products and processes. In environmental testing, organisms and their toxins that indicate the pollution of an environmental medium, e.g. soil, water, air, etc., are analyzed. Food testing includes the quantitation of organisms, e.g. bacteria, fungi, etc., as a means of quality control. In industrial process monitoring, nucleic acids are detected and/or quantified to indicate proper control of a production process and/or to generate a signal if such processes are out of control. In insurance testing, organisms and/or their toxins are identified in screening tests to determine the risk category of a client or to help approve candidates. There are various other applications of the detection and/or quantitation of nucleic acids and new applications are being developed constantly.

The most common techniques to detect and measure nucleic acid analytes are based on the sequence-specific hybridization of the analyte with a complimentary nucleotide sequence probe which is marked with a detectable label, typically a fluorescent label, a radioactive label or another chemical label that can be detected in a secondary reaction. The probe is combined with the nucleic acid analyte, either in situ as part of a biological system or as isolated DNA or RNA fragments. The hybridization conditions are those that allow the probe to form a specific hybrid with the analyte, while not becoming bound to non-complementary nucleic acid molecules. The analyte can be either free in solution or immobilized on a solid substrate. The probe's detectable label provides a means for determining whether hybridization has occurred and thus, for detecting the nucleic acid analyte. The signal that is generated via the detectable sample can in some instances be measured quantitatively to back-calculate the quantity and the concentration of the analyte.

Current methods used to detect and measure nucleic acid analytes are briefly described below.

PCR Methods

The polymerase chain reaction (PCR) amplification of nucleic acids is regularly performed using fluorescently labeled oligonucleotide primers to produce an amplified DNA product that can be detected and quantified absolutely. A wide range of fluorochromes are now commercially available with spectral characteristics (λₘₐₓ excitation and λₘₐₓ emission) covering wavelengths in the range of 350 to 700 nm, and into the near infra-red region of the electromagnetic spectrum. Thus, simultaneous multiple detection of labeled molecules can be performed on the same sample, for example, following ‘multiplex’ PCR amplification of several nucleic acid sequences using pairs of oligonucleotide primers labeled with different fluorophores. Each pair gives rise to a separate amplified product that can be unambiguously identified due to its fluorescent label.

FISH Methods

Fluorescent in situ hybridization (FISH) is an important tool for clinical diagnosis and gene mapping. Labeled nucleic acid probes are used with multiple, simultaneous fluorescent detection to locate specific nucleic acid sequences in cells and tissues, and with the number of fluorochromes available there is the potential to visualize several fluorescent signals relating to different genetic sequences within the same sample.

Nucleic Acid Microarrays

Microarrays of nucleic acids that are prepared by combinatorial chemistry methods provide a convenient means to assay multiple analytes, up to tens of thousands, simultaneously. Typically, microarrays are probed with fluorescently labeled nucleic acid species, for example, from a clinical sample, and the position of any hybridized, labeled nucleic acid is identified using fluorescence microscopy. The position relates to a known nucleic acid sequence immobilized at that part of the microarray.

Fluorescence Energy-Transfer (FRET) Methods

FRET relies upon the interaction of a fluorescent donor dye and a fluorescent acceptor dye, both of which are either attached to the same molecule or to different molecules. If the donor and acceptor dyes are in proximity to one another, the acceptor dye quenches the fluorescent signal of the donor dye upon excitation. However, when the two dyes are held apart from one another, the fluorescence of the donor dye can be detected.

Molecular Beacon Methods

Molecular beacons are nucleic acid probes that contain both a covalently attached fluorescent dye and a non-fluorescent quencher moiety. Molecular beacons allow the diagnostic detection of specific nucleic acid sequences through their structural characteristics. The probes possess hairpin-forming regions, and in the absence of a complementary nucleic acid strand, the fluorescent dye and the quencher are in close proximity to one another and quenching of the fluorescent signal results. When incubated with a target nucleic acid analyte that possesses a complementary sequence, the probe anneals to the target, such that the fluorescent dye and the probe are held apart from one another, and fluorescence can be detected signifying the presence of a particular nucleic acid sequence.

Preferably, methods to detect and/or quantify nucleic acid analytes are carried out as homogeneous assays, which require no separate analyte manipulation or post-assay processing. Classically, agarose gel electrophoresis, possibly followed by Southern-blot hybridization or enzyme-linked immunoassays was used to detect and quantitate nucleic acid. Maniatis et al. (1982) “Molecular Cloning: A Laboratory Manual,” Cold Spring Harbor Laboratory Press, NY, which is incorporated herein by reference in its entirety. Such procedures, as well as, other methods that similarly rely on end-point analysis are generally labor intensive, require several separate and distinct handling processes and skilled personnel, are relatively slow to produce a result, and are prone to contamination and false positives due to the open system. In comparison, the advantages of a homogeneous assay, which represents a fully enclosed homogenous real-time detection system, include a faster turn-around time, especially when using microvolumes, higher throughput, better options for automation and multi-parallel analysis, and the use of a fully enclosed test system, which reduces the likelihood of contamination.

Homogeneous assays are particularly desirable with PCR methods and other amplification methods, because the amplification and the detection of the nucleic acid analyte can be carried out in one step without any risk of cross-contamination, which is a severe problem with all methods that rely on extensive amplification, especially in high-throughput analysis labs.

Prior art homogeneous detection and quantification methods for nucleic acid analytes involve a variety of chemistries and formats, which are exemplified below.

Hydrolysis Probes

Hydrolysis probes are described in Holland and Gelfand (1991) Proc. Natl. Acad. Sci. USA 88:7376-80 and U.S. Pat. No. 5,210,015. Each of these references is specifically incorporated herein by reference in its entirety. This method takes advantage of the 5′-exonuclease activity present in the thermostable Taq DNA polymerase enzyme used in PCR (Taqman™ technology, Perkin-Elmer Applied Biosystems, Foster City, Calif., USA) and is applied to homogeneous detection in PCR, as described by Heid et al. (1996) Genome Methods 6:986-94, which is incorporated herein by reference in its entirety. This method involves the use of a nucleic acid probe, which is labeled with a fluorescent detector dye and an acceptor dye. Typically, the two dyes are attached to the 5′- and 3′-termini of the probe and when the probe is intact, the fluorescence of the detector dye is quenched by fluorescence resonance energy transfer (FRET). The probe anneals downstream of the amplification target site on the template DNA in PCR reactions. Amplification is directed by standard primers upstream of the probe, using the polymerase activity of the Taq enzyme. FRET quenching continues until the Taq polymerase reaches the region where the labeled probe is annealed. Taq polymerase recognizes the probe-template hybrid as a substrate, hydrolyzing the 5′ detector dye during primer-directed DNA amplification. The hydrolysis reaction releases the quenching effect of the quencher dye on the reporter dye, thus resulting in increasing detector fluorescence with each successive PCR cycle.

Mixed RNA/DNA sequence probes can also serve as hydrolysis probes to monitor PCR reactions, as described by Winger et al., U.S. Pat. No. 6,251,600 B1, which is incorporated herein by reference in its entirety. The mixed RNA/DNA probes contain blocks of DNA nucleotides at either end of the probe and a stretch of RNA nucleotide sequence between the flanking DNA blocks. This type of probe also contains a detector and an acceptor dye, which are attached to the respective DNA blocks of the probe. Upon hybridization to a nucleic acid analyte, the resulting hybrid contains two stretches of DNA:DNA duplex structure, flanking a stretch of DNA:RNA duplex structure. In the presence of the enzyme RNAse H, the DNA:RNA duplex structure is subject to cleavage, because RNAse H specifically recognizes DNA:RNA duplexes and cleaves the RNA portion of these duplexes. As a result the two blocks of DNA nucleotide sequence of the probe are separated, which in turn results in an increased fluorescence of the detector dye, which is no longer quenched by the acceptor.

Hairpin Probes

Hairpin probes or Molecular Beacons™ are described by Tyagi et al. (1996) Nat. Biotechnol. 14:303-308, and are applied to homogeneous detection in PCR, as described by Marras et al. (1999) Genetic Analysis 14:151-156, each of which is incorporated herein by reference in its entirety. Molecular beacons are nucleic acid probes that are able to form a hairpin substructure due to the presence of two inverted-repeat sequences. They carry covalently attached detector and quencher dyes at the end of both arms of the hairpin substructure of the probe. This design allows for self-complementary hybridization of the two inverted repeat sequences to form a stable, hairpin structure in the absence of a specific target. The detector and quencher dyes are in close proximity to one another in this conformation, which results in quenching of the detector fluorescence. The stretch of nucleotide sequence between the inverted repeat sequences of a molecular beacon is complementary to its target, thus directing specific probe-target hybridization, which results in efficient separation of the quencher dye from the detector dye with subsequent light emission. Thus, in the presence of a complementary target sequence, the hairpin structure is eliminated and the separated dye fluoresces. No overlap is required between the emission spectrum of the fluorophore and the absorption spectrum of the quencher. This allows for a wider range of fluorophores to be used in molecular beacons as compared with hydrolysis probes (TaqMan™). Hairpin probes are most commonly used as “free-floating” probes to detect amplicons as they are produced by standard PCR amplification, but can also be attached to one of the primers to act as a self-probing beacon as described by Whitcombe et al. (1999) Nat. Biotechnol. 17:804-807, which is incorporated herein by reference in its entirety.

Hybridization Probes

Hybridization probe design entails the use of two sequence-specific nucleic acid probes, each labeled with a fluorescent dye, one dye being a donor dye, the other dye being an acceptor dye. Typically, the two probes are designed to hybridize to a nucleic acid analyte close to each other in a head-to-tail arrangement that brings the two dyes into close proximity. In this arrangement, as demonstrated by Cardullo et al. (1988) Proc. Natl. Acad. Sci. USA 85:8790-04, which is incorporated herein by reference in its entirety, the fluorescence of the acceptor dye is enhanced if the donor is excited due to the radiationless uptake of energy from the donor. This method is applicable to PCR reactions (LightCycler™ technology, Roche Diagnostics, Indianapolis, Ind., USA), as demonstrated by e.g. Espy et al. (2000) J. Clin. Microbiol. 38:795-799, which is incorporated herein by reference in its entirety. For use with the LightCycler™ instrument of Roche Diagnostics the 3′-end of one probe is labeled with fluorescein as a donor and the 5′-end of the other probe can be labeled with LC Red 640 or LC Red 705 as an acceptor. Upon the occurrence of FRET between the donor and the acceptor, the fluorescence of the acceptor is detected. The transfer of fluorescent resonance energy only occurs when both probes hybridize to the target in very close proximity, the optimal distance being one to five intervening bases between probes. Hybridization probes are used in conjunction with standard primers to direct the PCR amplification.

The described fluorescence based methods are all limited in that they lack specificity and discrimination capability e.g. towards certain types of mutations. Thus, they can not cope with the growing demand for methods allowing the rapid screening of complete genomes for mutations, particularly single base mutations, in a high-throughput format. With regard to the increasing importance of SNPs and their analysis, e.g. for medical diagnosis, such methods relying on fast fluorogenic techniques are highly desirable.

The nucleic acid probes described herein, as well as, the related methods of the invention offer solutions for these requirements, by combining fluorogenic detection systems with the exceptional discrimination capability of locked nucleic acids (LNA).

LNA are a novel class of nucleic acid analogues not occurring in nature, which have been described by Wengel et al. (1999) WO 99/14226, which is incorporated herein by reference in its entirety. Monomeric LNA moieties contain a methylene bridge that connects the 2′-oxygen with the 4′-carbon of ribose, resulting in a bicyclic compound as illustrated by the following formula:

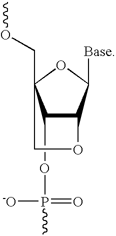

Oligonucleotides containing LNA are readily synthesized by standard phosphoramidite chemistry. Furthermore, standard methods for attaching a variety of linkers, modifiers, fluorescent labels and other reporter groups can easily be adopted to synthesize respective derivatives of such oligonucleotides, comprising either LNA only or LNA in combination with DNA and/or RNA.

As discussed in the Wengel et al. reference (WO 99/14226), duplexes of oligonucleotides comprised of LNA and DNA/RNA or LNA alone, with complementary DNA or RNA exhibit very high thermal stabilities, while obeying the Watson-Crick base pairing rules. In general, the thermal stability of such heteroduplexes is increased by 3 to 8° C. per monomeric LNA moiety in the duplex. Oligonucleotides containing LNA can be used as primers in PCR reactions resulting in a higher discrimination towards single base mutations in the template nucleic acid compared to normal DNA primers.

The instant invention describes novel fluorescence based methods to detect and/or quantify nucleic acid analytes. Included in the present invention are novel nucleic acid probes and pairs of nucleic acid probes. The methods and probes of this invention have significant advantages and do not suffer from the limitations inherent in the prior art methods and probes. The nucleic acid probes described in this invention carry at least one fluorescent dye and comprise one or more monomeric LNA moieties. They are highly sequence specific and lead to improved discrimination in genotyping assays. They can easily be adopted in homogeneous assays, in particular in PCR based assays, and provide the results of the assays in real time. The probes and pairs of probes are amendable to multiplexing in such assays, and are also applicable in assays conducted on nucleic acid microarrays.

Probes comprising LNA moieties that contain a fluorophor moiety and a quencher moiety are described by Jakobsen et al. (2002) EP 1247815, which is incorporated herein by reference in its entirety. However, Jakobsen et al. do not disclose how to use their invention and completely fail in reducing it to practice. Furthermore, the design of the probes described by Jakobsen et al. is very limited in that at least the second mono-nucleotidic position from the 3′- and/or the 5′-end has to be a LNA moiety.

### SUMMARY OF THE INVENTION

The present invention includes novel methods for detecting or quantifying nucleic acid analytes through their interactions with a nucleic acid probe or a pair of nucleic acid probes. In one embodiment, the method of the present invention comprises the steps of: a) providing a nucleic acid probe, wherein said nucleic acid probe is comprised of at least one monomeric LNA moiety and two or more non-identical covalently attached dyes, wherein at least one dye is fluorescent; b) contacting said nucleic acid probe with a nucleic acid analyte so as to allow for the hybridization of the nucleic acid probe with the nucleic acid analyte; and c) measuring the change in the fluorescence of the nucleic acid probe, wherein said change in fluorescence is related to the hybridization of the nucleic acid probe with the nucleic acid analyte; whereby the presence or amount of the analyte is determined.

In another embodiment, the method of the present invention comprises the steps of: (a) providing a pair of nucleic acid probes, wherein each probe of said pair differ in their nucleic acid sequence, and wherein said pair collectively include at least one monomeric LNA moiety and are collectively derivatized with two or more non-identical covalently attached dyes, wherein at least one dye is fluorescent, and wherein each probe of said pair is derivatized with at least one of said dyes; (b) contacting said pair of nucleic acid probes with a nucleic acid analyte so as to allow for the hybridization of the pair of nucleic acid probes with the nucleic acid analyte in such a way that both probes are hybridized to adjacent segments of the target sequence of the nucleic acid analyte; and (c) measuring the change in the fluorescence of the pair of nucleic acid probes, wherein said change in fluorescence is related to the hybridization of the nucleic acid probe pair with the nucleic acid analyte; whereby the presence or amount of the analyte is determined.

Included in the present invention are novel nucleic acid probes for use in the method of the invention. The novel nucleic acid probes of the invention are comprised of an n-meric nucleic acid comprising any number of 1 to n monomeric locked nucleic acid (LNA) moieties that may be situated in any position(s) of the nucleic acid sequence. In one embodiment, n is an integer selected from 1-1000. In a preferred embodiment, n is an integer selected from 10-200. The nucleic acid probes are further characterized in that they are derivatized with one or more dyes, wherein said dyes are independently selected from either fluorescent dyes or non-fluorescent quencher dyes. The methods provided by the invention are based on the change of fluorescence upon hybridization of the inventive nucleic acid probes or pairs of nucleic acid probes with a nucleic acid analyte. Due to the fact that the inventive nucleic acid probes hybridize to analytes with increased specificity and affinity, these methods represent an improvement over the prior art.

### BRIEF DESCRIPTIONS OF THE DRAWINGS

FIG. 1A displays the observed relative fluorescence intensities in a real-time PCR experiment as a function of the PCR cycle number using nucleic acid probe pair 1 and DNA templates related to the human cystic fibrosis SNP G542X. Nucleic acid probe pair 1 (Table 1) is comprised of deoxynucleotides only. The corresponding experiments are described in Example 4. The plots for the wild type template, the heterozygous template, the mutant type template and the control without template are represented by the lines I, II, III and IV, respectively.

FIGS. 1B to 1D display the observed relative fluorescence intensities in real-time PCR experiments as a function of the PCR cycle number using nucleic acid probe pairs 2, 3 and 4, respectively, and DNA templates related to the human CF SNP G542X. Nucleic acid probe pairs 2, 3 and 4 (Table 1) have 3 or 4 monomeric LNA moieties in each probe. The corresponding experiments are described in Example 4. The plots for the wild type template, the heterozygous template and the mutant type template are represented by the lines I, II and III, respectively.

FIGS. 1E and 1F display the observed relative fluorescence intensities in real-time PCR experiments as a function of the PCR cycle number using nucleic acid probe pairs 5 and 6, respectively, and DNA templates related to the human CF SNP G542X. The corresponding experiments are described in Example 4. Both probe pairs are identical in length and sequence, but the probes of probe pair 5 are comprised of 5 monomeric LNA moieties each, whereas the probes of probe pair 6 are comprised of deoxynucleotides only.

FIGS. 2A to 2F depict the time derivatives of the melting curves for the probe pairs 1 to 6, respectively, as measured subsequent to the corresponding PCR experiments according to Example 4 with amplicons derived from DNA templates related to the human CF SNP G542X. The plots of the derivative melting curves for the wild type derived amplicon, the heterozygous type derived amplicon, the mutant type derived amplicon and the control are represented by the lines I, II, III and IV, respectively.

FIG. 3A is identical to FIG. 1A and is added for purposes of comparison only.

FIGS. 3B to 3D depict the observed relative fluorescence intensities in real-time PCR experiments as a function of the PCR cycle number using nucleic acid probe pairs 7, 8 and 9, respectively, and DNA templates related to the human CF SNP G542X. Nucleic acid probe pairs 7, 8 and 9 are comprised of 3 or 4 monomeric LNA moieties in the probe carrying the donor dye only. The corresponding experiments are described in Example 5. The plots for the wild type template, the heterozygous template, the mutant type template and the control without template are represented by lines I, II, III and IV, respectively.

FIGS. 3E and 3F depict the observed relative fluorescence intensities in real-time PCR experiments with the nucleic acid probe pairs 10 and 11, respectively, and DNA templates related to the human CF SNP G542X as a function of the PCR cycle number. The corresponding experiments are described in Example 5. Both probe pairs are identical in length and sequence, but the Red 640 derivatized probe of probe pair 10 is comprised of 5 monomeric LNA moieties, whereas the probes of probe pair 11 are comprised of deoxynucleotides only. The plots for the wild type template; the heterozygous template, the mutant type template and the control without template are represented by the lines I, II, III and IV, respectively.

FIGS. 4A to 4F display the time derivatives of the melting curves for the probe pairs 1 and 7 to 11, respectively, measured according to Example 5 subsequent to the PCR experiments described in Example 5 with nucleic acid templates related to the human CF SNP G542X. The plots of the derivative melting curves for the wild type derived amplicon, the heterozygous type derived amplicon, the mutant type derived amplicon and the control are represented by the lines I, II, III and IV, respectively.

### DETAILED DESCRIPTION OF THE INVENTION

The present invention includes novel methods for detecting or quantifying nucleic acid analytes through their interactions with a nucleic acid probe or a pair of nucleic acid probes, wherein the probe or the pair of probes is comprised of at least one monomeric LNA moiety and two or more dyes, wherein at least one of said dyes is fluorescent. Preferably the probe or the pair of probes is comprised of a combination of two dyes, wherein either both are fluorescent dyes that coactively function as the donor dye and the acceptor dye of a FRET system, or wherein one of said dyes is a fluorescent dye and the other is a corresponding non-fluorescent quencher dye.

Included in the present invention are novel nucleic acid probes for use in the detection and quantification of analytes according to the methods of the invention. The novel nucleic acid probes of the invention are comprised of an n-meric nucleic acid comprising any number of 1 to n monomeric locked nucleic acid (LNA) moieties that may be situated in any position(s) of the nucleic acid sequence. The nucleic acid probes are further characterized in that they are derivatized with one or more dyes, wherein said dyes are independently selected from fluorescent dyes or non-fluorescent quencher dyes.

The methods provided by the invention are based on the change of fluorescence resulting from the hybridization of the inventive nucleic acid probes or pairs of nucleic acid probes with nucleic acid analytes. Due to the fact that the inventive probes and pairs of probes hybridize to analytes with increased specificity and affinity, these methods represent an improvement over the prior art.

Various terms are used herein to refer to aspects of the present invention. To aid in the clarification of the description of the components of the invention, the following descriptions are provided.

It is to be noted that the term “a” or “an” entity refers to one or more of that entity; for example, a nucleic acid that carries a multitude of dyes refers to one or more nucleic acids that carry a multitude of dyes. As such, the terms “a” or “an,” “one or more” and “at least one” are used interchangeably herein.

The term “analyte” refers to a nucleic acid molecule or a mixture of nucleic acid molecules, as defined below, that is to be detected or quantified using the method of this invention. The terms “target nucleic acid analyte” and “nucleic acid analyte” are used interchangeably with the term analyte in the context of this invention.

As used herein, “nucleic acid” means either DNA, RNA, single-stranded or double-stranded and any chemical modifications thereof, such as PNA and LNA. Nucleic acids can be of any size and are preferably oligonucleotides. Modifications include, but are not limited to, those that provide other chemical groups that incorporate additional charge, polarizability, hydrogen bonding, electrostatic interaction, and functionality to the individual nucleic acid bases or to the nucleic acid as a whole. Such modifications include, but are not limited to, modified bases such as 2′-position sugar modifications, 5-position pyrimidine modifications, 8-position purine modifications, modifications at cytosine exocyclic amines, substitution of 5-bromo-uracil; backbone modifications, methylations, unusual base-pairing combinations such as the isobases isocytidine and isoguanidine and the like. Modifications can also include 3′ and 5′ modifications such as capping. The nucleic acid can be derived from a completely chemical synthesis process, such as a solid phase mediated chemical synthesis, or from a biological origin, such as through isolation from almost any species that can provide DNA or RNA, or from processes that involve the manipulation of nucleic acids by molecular biology tools, such as DNA replication, PCR amplification, reverse transcription, or from a combination of those processes. Virtually any modification of the nucleic acid and nucleic acids of virtually any origin are contemplated by this invention.

“Covalently attached” in the context of this invention describes an attachment of one molecular moiety to another molecular moiety through covalent chemical bonds, i.e. chemical bonds that are established through the pairing of electrons from the atoms that are bonded together.

A “dye” in the context of this invention is any organic or inorganic molecule that absorbs electromagnetic radiation at a wavelength greater than or equal to 340 nm.

A “fluorescent dye” as defined herein is any dye that emits electromagnetic radiation of longer wavelength by a fluorescence mechanism upon irradiation by a source of electromagnetic radiation, including but not limited to a lamp, a photodiode or a laser. Fluorescent dyes may also be referred to as fluorophores. Any known fluorescent dyes are contemplated for use within the context of this invention. Examples of known fluorescent dyes can be found for example in Haugland, Handbook of Fluorescent Probes and Research Products, (9^{th} Ed.). Specific, examples of fluorescent dyes include, but are not limited to fluorescein.

A “quenching group” or “quencher moiety” as defined herein is a dye that reduces the emission of fluorescence of another dye. Thus, illumination of a fluorescent dye in the presence of a quenching group leads to an emission signal that is less intense than expected. The reduction of fluorescence emission, also referred to herein as quenching, occurs through energy transfer between the fluorescent dye and the quenching group. This can be caused by a radiationless energy transfer through space (Fluorescence Resonance Energy Transfer (FRET)), see Yang et al. (1997) Methods Enzymol. 278:417-44, which is incorporated herein by reference in its entirety, or by the formation of ground state heterodimers, see Bernacchi et al. (2001) Nucleic Acids Res. 29:e62, which is incorporated herein by reference in its entirety, or by other mechanisms.

A “nucleic acid probe” as defined herein is a nucleic acid that carries or is derivatized with one or more covalently attached dyes, wherein said dyes are independently selected from fluorescent dyes or non-fluorescent quenching dyes. In a preferred embodiment, a nucleic acid probe contains either two covalently attached dyes, or as part of a pair of nucleic acid probes one covalently attached dye.

As used herein, “fluorescence resonance energy transfer” or “FRET” refers to a radiationless energy transfer phenomenon in which the light emitted by the excited fluorescent dye is absorbed at least partially by a quenching group. The quenching group can either radiate the absorbed light as light of a different wavelength or it can dissipate it as heat. FRET depends on an overlap between the emission spectrum of the fluorescent dye and the absorption spectrum of the quenching group. FRET also depends on the distance between the quenching group and the fluorescent group. Above a certain critical distance, the quenching group is unable to absorb the light emitted by the fluorescent group, or can do so only poorly. FRET is described in detail in Yang et al. (1997) Methods Enzymol. 278:417-444.

A “donor” as defined herein is a dye that is part of a FRET system in which the dye transfers energy to another dye by a radiationless process. Generally, in such a system the fluorescence of the dye decreases when it is part of a FRET system. An example of a donor dye is the dye fluorescein.

An “acceptor” as defined herein is a dye that is part of a FRET system in which the dye accepts energy from another dye by a radiationless process. Generally, in such a system the fluorescence of the acceptor dye increases when excited at the wavelength of the corresponding donor of the FRET system as compared to the fluorescence of the acceptor dye when it is not part of a FRET system, see Yang et al. (1997) Methods Enzymol. 278:417-444. An example of a donor dye is the dye LC Red 604.

A “homogeneous assay” as defined herein is a process to detect or quantify a nucleic acid analyte that requires no separate analyte manipulation or post-assay processing to record the result of the assay. Homogeneous assays are carried out in closed tubes, meaning that no further addition of reagents or supplementary chemicals is necessary to record the result once the assay is started. Homogeneous assays allow recordation of the result of the assay in real time, meaning that the result of the assay can be continuously recorded as the assay progresses in time.

In one embodiment, the present invention includes a method for the detection or quantification of a nucleic acid analyte comprising the steps of: (a) providing a nucleic acid probe, wherein said nucleic acid probe is comprised of at least one monomeric LNA moiety and two or more non-identical covalently attached dyes, wherein at least one of said dyes is fluorescent; (b) contacting said nucleic acid probe with a nucleic acid analyte so as to allow for the hybridization of the nucleic acid probe with the nucleic acid analyte; and (c) measuring the change in the fluorescence of the nucleic acid probe, wherein said change in fluorescence is related to the hybridization of the nucleic acid probe with the nucleic acid analyte; whereby the presence or amount of the analyte is determined.

In a preferred embodiment, said change in fluorescence of the nucleic acid probe occurs upon the hybridization of the nucleic acid probe with the nucleic acid analyte. This embodiment of the present invention is exemplified by a nucleic acid probe, which functions similarly to an aforementioned molecular beacon. In addition to including one or more monomeric LNA moieties and a covalently attached fluorescent dye, such a probe may also be comprised of a covalently attached non-fluorescent quencher moiety, providing for an increase in fluorescence upon specific annealing to the target sequence of an analyte.

In another preferred embodiment of the present invention, said change in fluorescence of the nucleic acid probe occurs upon the hydrolysis of the nucleic acid probe that is hybridized to the nucleic acid analyte. A particularly preferred method according to this embodiment, is the aforementioned real-time assay using hydrolysis probes which, subsequent to annealing to their target sequence, are hydrolyzed in the course of the amplification step of the PCR, due to the additional 5′-exo nuclease activity of the polymerase employed. Particularly useful in this regard are Taqman™ analogous probes comprising, in addition to one or more monomeric LNA moieties, e.g. a fluorescent dye such as fluorescein and a non-fluorescent quencher dye such as TAMRA (carboxy tetramethyl rhodamine), which produce an increase in the fluorescent signal with the progressing amplification.

In another embodiment, the present invention includes a method for the detection or quantification of a nucleic acid analyte comprising the steps of: (a) providing a pair of nucleic acid probes, wherein each probe of said pair differ in their nucleic acid sequence, and wherein said pair collectively include at least one monomeric LNA moiety and are collectively derivatized with two or more non-identical covalently attached dyes, wherein at least one dye is fluorescent, and wherein each probe of said pair is derivatized with at least one of said dyes; (b) contacting said pair of nucleic acid probes with a nucleic acid analyte so as to allow for the hybridization of the pair of nucleic acid probes with the nucleic acid analyte in such a way that both probes are hybridized to adjacent segments of the target sequence of the nucleic acid analyte; and (c) measuring the change in the fluorescence of the pair of nucleic acid probes, wherein said change in fluorescence is related to the hybridization of the nucleic acid probe pair with the nucleic acid analyte; whereby the presence or amount of the analyte is determined.

In a preferred embodiment, the nucleic acid probes of said probe pairs are comprised of one dye and hybridize to the analyte side-by-side in a head-to-tail arrangement. A particularly preferred method according to this embodiment, is the aforementioned real-time assay using hybridization probes of the invention that are analogous to the LightCycler™ probes, wherein one probe of the respective probe pairs is derivatized with a donor dye, such as fluorescein and the other probe is derivatized with an acceptor dye, such as LC Red 640. With the growing quantity of amplicons generated in the course of the progressing PCR reaction an increasing number of hybridization probes anneal pairwise to the target sequence in the annealing step of the PCR, resulting in the build-up of the FRET system and consequently in an enhanced fluorescence of the acceptor dye.

The present invention includes the nucleic acid probes employed in the methods of the invention. The novel nucleic acid probes of the invention are comprised of an n-meric nucleic acid comprising any number of 1 to n monomeric locked nucleic acid (LNA) moieties that may be situated in any position(s) of the nucleic acid sequence. In one embodiment, n is an integer selected from 1-1000. In a preferred embodiment, n is an integer selected from 10-200. The nucleic acid probes are further characterized in that they are derivatized with one or more dyes, wherein said dyes are independently selected from either fluorescent dyes or non-fluorescent quencher dyes.

The nucleic acid probes of the invention can be readily prepared by applying known methods for solid phase oligonucleotide assembly and methods for conjugating reporter molecules. The introduction of monomeric LNA moieties at any desired position in the oligonucleotide sequence can be easily accomplished by introducing the corresponding LNA phosphoramidites into the synthetic scheme of solid phase oligonucleotide synthesis, as extensively reviewed by Beaucage et al. (1992) Tetrahedron 48:2223-2311, which is incorporated herein by reference in its entirety. Since LNA phosphoramidites possess very similar properties in regard to all steps of oligonucleotide synthesis, mixed oligomers comprising DNA and/or RNA as well as LNA can be prepared using standard protocols of automated solid phase synthesis that may have to be adapted only slightly at the most. Also, oligonucleotides comprising LNA are commercially available, e.g. from Proligo LLC (Boulder, Colo., USA).

The covalent attachment of dyes to nucleic acids can be achieved by a variety of methods known to those of skill in the art. The covalent attachment of dyes to nucleic acids is reviewed in Davies et al. (2000) Chem. Soc. Rev. 29:97-107, which is incorporated herein by reference in its entirety. Examples include, but are not limited to incorporation of the dyes during the synthesis of nucleic acids, typically solid phase synthesis, post-synthetic labeling of either synthetic nucleic acids or nucleic acids derived through enzymatic reactions, e.g. the PCR reaction, and enzymatic methods of incorporation of dyes into nucleic acids, e.g. the use of dye conjugated deoxynucleotide triphosphates in primer elongation reactions such as a PCR reaction.

Methods for introducing dyes into oligonucleotides using solid phase synthetic methods are well established and many related reagents are commercially available. The incorporation of dyes to the 5′-end of an oligonucleotide entails the conversion of the dyes into their phosphoramidite derivatives, which are then employed in the phosphoramidite solid phase synthetic method similar to nucleoside phosphoramidites, as reviewed by Beaucage et al. (1993) Tetrahedron 49:1925-63, which is incorporated herein by reference in its entirety. For the incorporation of dyes to the 3′-end of oligonucleotides, solid supports functionalized with various dyes have been described and are in part commercially available, as reviewed e.g. by Davies et al. (2000) Chem. Soc. Rev. 29:97-107. Briefly, the oligonucleotide is assembled on a linker moiety, which carries the dye and is also connected to the solid support via a cleavable bond. After completion of the oligonucleotide assembly on the support the 3′-labeled oligonucleotide is released from the support in the standard cleavage/deprotection step, which may have to be slightly modified due to the limited stability of some dyes to basic conditions.

Additionally, a further group of functionalized solid supports allow the synthesis of 3′-phosphorylated oligonucleotides directly in the course of solid phase syntheses. These special supports are reviewed by Beaucage et al. (1993) Tetrahedron 49:10441-10488. A representative example is the so-called phosphate-on CPG, which features facile handling and mild cleavage/deprotection conditions. This support can be obtained from Proligo LLC (Boulder, Colo., USA). The 3′-phosphorylation is very useful for preparing hybridization probes that carry a dye at the 5′-end, because the 3′-phosphate groups inhibit the undesired enzymatic elongation of the probe.

Post-synthetic labeling of synthetic nucleic acids or nucleic acids derived from enzymatic reactions involves the incorporation of a functional group or groups into the nucleic acids to serve as anchor points for the attachment of one or more dyes. The dyes are then derivatized with a chemical group or moiety, which will react with a functional group of the nucleic acid to promote the formation of a covalent bond between the nucleic acid and the dye. The functional group incorporated into the nucleic acid is selected from any group that is capable of reacting selectively with the group or moiety that is incorporated into the dye. Examples of such functional groups which can be incorporated into nucleic acids and groups or moieties which can be incorporated into dyes, include, but are not limited to, amino groups/activated esters, e.g. hydroxysuccinimide esters; thiol groups/electrophilic groups; and dienes/dienophiles, e.g. maleimides. Methods known to those skilled in the art to promote a covalent bond between a nucleic acid and a dye are reviewed by Grimm et al. (2000) Nucleosides & Nucleotides 19:1943-65, which is incorporated herein by reference in its entirety.

The incorporation of functional groups into synthetically derived nucleic acids can be achieved using a variety of methods. A standard method known to those skilled in the art is the use of linker phosphoramidites during solid phase synthesis. Linker molecules useful in the solid phase phosphoramidite method consist of an amidite-moiety, a spacer and a functional group that is protected if the functional group interferes with the amidite synthesis. Prominent examples are linkers to introduce amino-functions or thiol-functions that can be introduced by a number of commercially available phosphoramidite linkers.

Example 1 describes the synthesis of two singly labeled nucleic acid probes that are well suited as a hybridization probe pair in applications of the present invention for monitoring PCR reactions. Both probes are prepared according to protocols employing standard phosphoramidite chemistry. The first probe, which is derivatized with the donor dye fluorescein, is assembled on a fluorescein functionalized CPG yielding the desired 3′-labeled oligonucleotide. The second probe, which is derivatized with the acceptor dye LC Red 604, is synthesized on a phosphate-on CPG to provide a probe that is finished with a 3′-phosphate group blocking the polymerase mediated extension of the probe during the PCR reaction. Following the assembly of the oligonucleotide on this support in a final coupling step a non-nucleotidic phosphoramidite containing an amino group is linked to oligonucleotide. The amino-functionalized oligonucleotide resulting from the cleavage/deprotection step is then reacted with a NHS ester derivatized acceptor dye LC Red 604, to provide a 5′-labeled probe that is blocked at the 3′-end by a phosphate group.

In a preferred embodiment the nucleic acid probes of this invention are comprised of either one dye attached at or close to the 3′- or the 5′-end of the nucleic acid, or two different dyes wherein one dye is attached to one end (3′- or the 5′-) of the nucleic acid and the second dye is attached at the other end of the nucleic acid, respectively. Particularly preferred are probes derivatized with a non-fluorescent quencher moiety at one end and a fluorescent dye at the other end of the nucleic acid. Such probes are very useful as hydrolysis probes in real-time PCR applications and may be regarded as Taqman™ analogous probes.

Also preferred herein are pairs of nucleic acid probes comprised of either: two nucleic acid probes each of which contains at least one monomeric LNA moiety or one nucleic acid probe including such LNA moieties together with a second nucleic acid probe that does not contain LNA. The nucleic acid probes of such a probe pair are further characterized in that they are complementary or largely complementary to adjacent segments of the target sequence of the analyte, and in that each probe of the probe pair is derivatized with at least one dye. Particularly preferred in this context are pairs of nucleic acid probes that are comprised of either: a fluorescent dye and a non-fluorescent quencher dye, or two fluorescent dyes that are able to jointly constitute the donor dye and the acceptor dye, respectively of a FRET system. Most preferred are the latter described nucleic acid probe pairs, wherein the donor and acceptor dyes are attached to the respective termini of the probes, which are then situated adjacent to each other after the annealing of the probes to the target sequence. Such nucleic acid probe pairs are very useful as hybridization probes in real-time PCR applications and may be regarded as analogous to pairs of LightCycler™ probes.

Also preferred are nucleic acid probes or pairs of nucleic acid probes having the above described properties, wherein said probes or probe pairs are complementary or largely complementary to section of a nucleic acid analyte comprising a SNP site, wherein a monomeric LNA moiety is positioned opposite to the SNP site subsequent to the hybridization of the probe with the analyte. The LNA moiety is then either complementary or is not complementary to the SNP site of the analyte. Such nucleic acid probes or probe pairs are particularly useful for genotyping applications.

In a preferred embodiment of the present invention, a nucleic acid probe or a pair of nucleic acid probes as described above, is used in homogeneous assays to detect or quantify nucleic acid targets. In such assays, a fluorescent signal is generated as a result of the presence of a complementary nucleic acid sequence in the analyte. The fluorescent signal is monitored and quantified with fluorescence detectors, including but not limited to fluorescence spectrophotometers, commercial systems that allow the monitoring of fluorescence in PCR reactions, e.g. instruments manufactured by Perkin-Elmer Applied Biosystems, Foster City, Calif., or LightCycler™ instruments manufactured by Roche Diagnostics, Indianapolis, Ind., or, in some instances, by the human eye.

In one embodiment, the homogeneous assay is conducted without the addition of reagents, other than buffers and other non-reactive ingredients. Such non-reactive ingredients include but are not limited to, EDTA, magnesium salts, sodium chloride, potassium chloride, inorganic phosphates, BSA (bovine serum albumin), gelatin, DMF, DMSO, urea, chaotropic salts or other non-reactive ingredients known to those skilled in the art, which are commonly employed in nucleic acid based diagnostic assays. In this embodiment of the invention, the nucleic acid probe or each probe of the pair of nucleic acid probes hybridize with a complementary nucleic acid sequence, if present in the target. This hybridization event entails the interaction of the dyes attached to the probe or the pair resulting in the generation of a fluorescent signal upon excitation.

Using appropriate target standards to generate concentration versus signal standard curves, the method of the invention can easily be used to quantitate the target. In addition to single stranded target nucleic acids, double stranded target nucleic acids can also be detected by the nucleic acid probe following denaturation. Targets that can be specifically detected and/or quantified using this method include, but are not limited to, plasmid DNA, cloning inserts in plasmid DNA, RNA transcripts, ribosomal RNA, PCR amplicons, restriction fragments, synthetic oligonucleotides, as well as any other nucleic acids and oligonucleotides.

Furthermore, depending on the design and of the nucleic acid probe or the pair of probes and the nature of their respective dyes, the fluorescent signal is either increased or decreased upon annealing to an analyte. For example, if one probe of a pair of nucleic acid probes is derivatized with a fluorescent dye and the other probe is derivatized with a non-fluorescent quencher dye, their head-to-tail hybridization to adjacent stretches of the target sequence results in a decrease of fluorescence upon excitation. However, a respective pair of nucleic acid probes comprising a fluorescent acceptor dye and a fluorescent donor dye, would result in an increase of the fluorescence of the acceptor dye upon hybridization and excitation of the donor dye.

In another particular embodiment of the invention, a homogeneous assay is conducted simultaneously with a PCR reaction. In this type of assay all components that are necessary to conduct a PCR reaction on the target nucleic acid analyte are added simultaneously with the nucleic acid probe or the pair of probes. The components of the PCR reaction include primers, a thermostable DNA polymerase, an aqueous buffer, magnesium chloride and deoxynucleotide triphosphates, and may also include other non-reactive ingredients, including, but not limited to, salts, BSA, gelatin, DMSO, chaotropic salts, as discussed above. As the PCR reaction progresses increasing amounts of double stranded PCR amplicons are formed which are denatured during the course of a PCR cycle. In these assays, the specific nucleic acid probe or probe pair contains a stretch of nucleic acid sequence that is complementary to a stretch of nucleic acid sequence on the formed amplicon. As a result of the hybridization of the nucleic acid probe or each probe of the pair of nucleic acid probes to its complementary stretch of nucleic acid sequence on the single stranded amplicon, a fluorescent signal is generated that is proportional to the amount of amplicon formed.

In another embodiment of the present invention, a nucleic acid probe or a pair of nucleic acid probes as described herein is employed in assays that are conducted on nucleic acid microarrays to detect or quantify nucleic acid targets. In such assays, a fluorescent signal is generated on a nucleic acid microarray depending on the presence of a complementary nucleic acid sequence in the analyte. Nucleic acid microarrays, also called nucleic acid chips, consist of ordered arrays of nucleic acids that are covalently attached to a solid surface, see Schena, ed., in DNA Microarrays A Practical Approach, Oxford University Press, and Marshall et al. (1998) Nat. Biotechnol. 16:27-31, each of which is specifically incorporated herein by reference in its entirety, for a comprehensive description of nucleic acid microarrays. The fluorescent signal generated in the assay can be monitored and quantified using fluorescence detectors, including but not limited to fluorescence imagers, e.g. commercial instruments supplied by Hitachi Corp., San Bruno, Calif. or confocal laser microscopes (confocal fluorescence scanners), e.g. commercial instruments supplied by General Scanning, Inc., Watertown, Mass. As discussed above, depending on the design and of the nucleic acid probe or the pair of probes and the nature of their dyes, either an increase or a decrease of fluorescence is observed upon hybridization.

In assays that are conducted on nucleic acid microarrays, the target nucleic acid analyte may be a mixture of nucleic acid sequences, consisting of up to hundreds of nucleic acid sequences, and in some instances of up to tens of thousands of nucleic acid sequences. This particularly applies to expression analysis, where many or all mRNA sequences that are present in a biological system, e.g. a certain cell type from a cell culture, are analyzed, see Hunt et al., eds., in Functional Genomics A Practical Approach, Oxford University Press, for a comprehensive description of expression analysis, which is specifically incorporated herein by reference in its entirety. Typically, the mRNA sequences are amplified by reverse transcription PCR with universal primers prior to their use as analytes in the assay. In this instance, all nucleic acid sequences present in the analyte are simultaneously applied to the microarray for analysis, thus allowing the interaction of all of the nucleic acid sequences of the analyte with all of the nucleic acids that are present on the array. In other instances, the target nucleic acid analyte contains a limited number of up to a hundred nucleic acid sequences and in some instances only one nucleic acid sequence. In this case, the limited number of sequences typically contain more than one stretch of specific nucleotide sequence to be analyzed, e.g. more than one single nucleotide polymorphism. The nucleic acid sequences may optionally be amplified by PCR with the aid of specific primers prior to their analysis on the microarray.

Generally, in analyses on microarrays, the fluorescent signals generated are converted to sequence specific results through the known relation of the location of a spot on the array and the nucleotide sequence attached to it.

In another embodiment, the methods of the invention are used to detect or quantify nucleic acid targets that are derived from genomic DNA in order to analyze for the presence or absence of polymorphisms in the genomic DNA. The polymorphisms can be deletions, insertions, or base substitutions or other polymorphisms of the genomic DNA. Typically, the polymorphisms are single nucleotide polymorphisms (SNPs), i.e. single base substitutions in the genomic DNA.

In a preferred embodiment, the genomic DNA is amplified in a PCR reaction using specific primers. The resulting amplicons contain the polymorphism(s) of interest, which are then assayed using one or more nucleic acid probes or pairs of probes that are complementary and/or partially complementary to the polymorphic site in such a manner that the polymorphic site can be identified in the assay. The assay is typically performed with probes having different sequences, which differ in one nucleotide corresponding to the polymorphic site and allow the discrimination of the possible variations at the polymorphic site upon hybridization of the amplicons. A sequence that is fully complementary will generate a fluorescent signal, whereas a sequence with a corresponding possible variation of the polymorphism, in many cases a single nucleotide variation, will not hybridize as efficiently as the fully complementary sequence under the conditions of the assay, and therefore will generate either a weaker fluorescent signal or no fluorescent signal at all. Typically several probes or pairs of probes are employed comprising more than one or all possible variations of nucleotide sequence corresponding to the polymorphic site of interest, e.g. both variations of a SNP, and therefore allows the detection and/or quantitation of more than one or all variations of the polymorphic site, e.g. both variations of a SNP. Therefore, in SNP detection typically both homozygote and heterozygote variations of the SNP can be detected. The above described method is highly amenable to be performed in a multiplexed format, e.g. by detecting different versions of a polymorphism simultaneously with probes or pairs of probes comprising the corresponding sequences and distinguishable fluorescent labels. Furthermore, in a similar manner more than one polymorphism can be assayed simultaneously.

This method can also be employed in an assay with a microarray that contains ordered spatially arranged nucleic acid probes in accordance with this invention. The nucleic acid probes contain stretches of nucleotide sequences that are complementary and/or partially complementary to the polymorphic sites in such a manner that the polymorphic sites can be identified in the assay.

Example 2 describes a general method for performing real-time PCR experiments using hybridization probe pairs and Example 3 describes a general procedure for measuring melting curves of probe pairs.

Example 4 describes real-time PCR analyses and subsequent melting curve measurements of a section of the human cystic fibrosis (CF) related transconductance regulator (CFTR) gene to examine the SNP site G542X using hybridization probe pairs of the invention. Cystic fibrosis is a prevalent and well-studied autosomal recessive disorder mainly affecting Caucasian populations at a frequency of about 0.05%. The cystic fibrosis CFTR gene that is altered in the disease is on chromosome 7, as described by Riordman et al. (1989) Science 245:1066-1073. Population screening has uncovered nearly 900 variants in the gene to date. Many of these are disease-causing mutations.

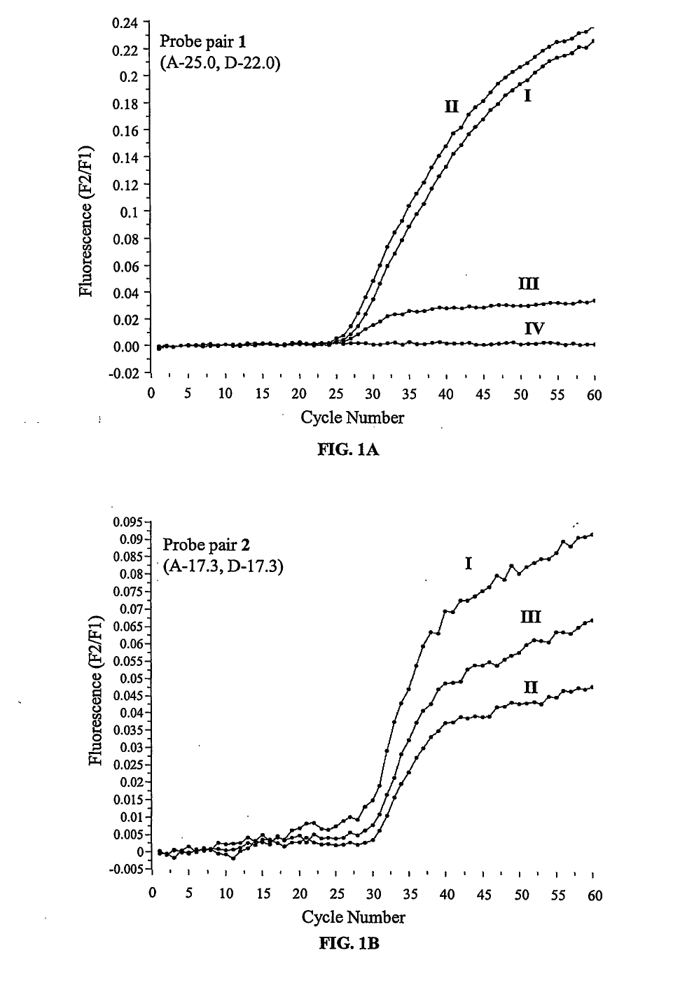

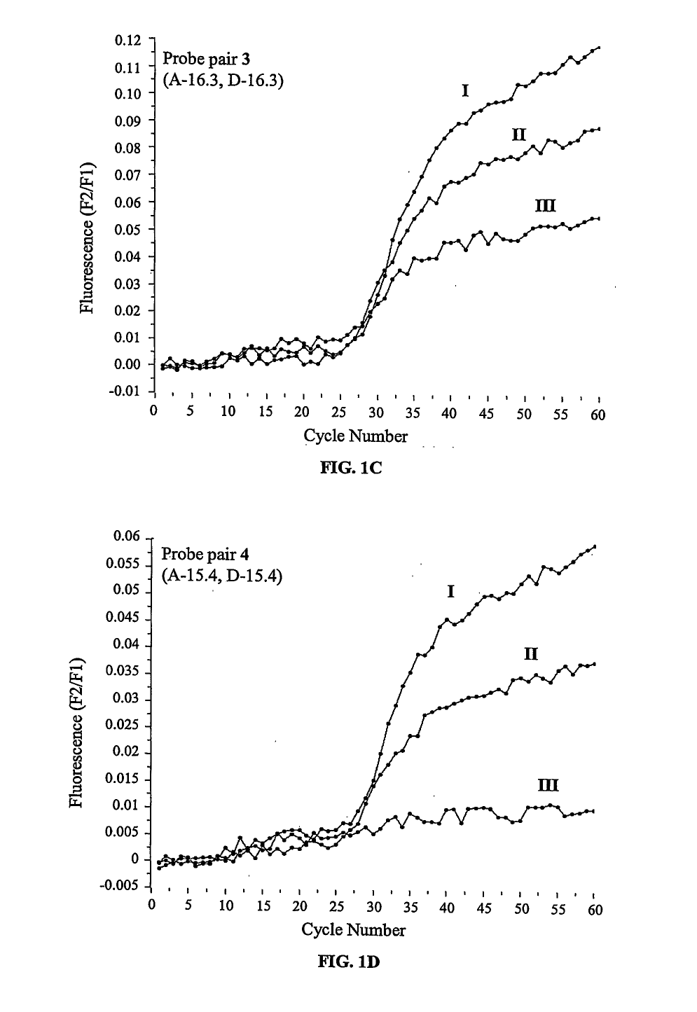

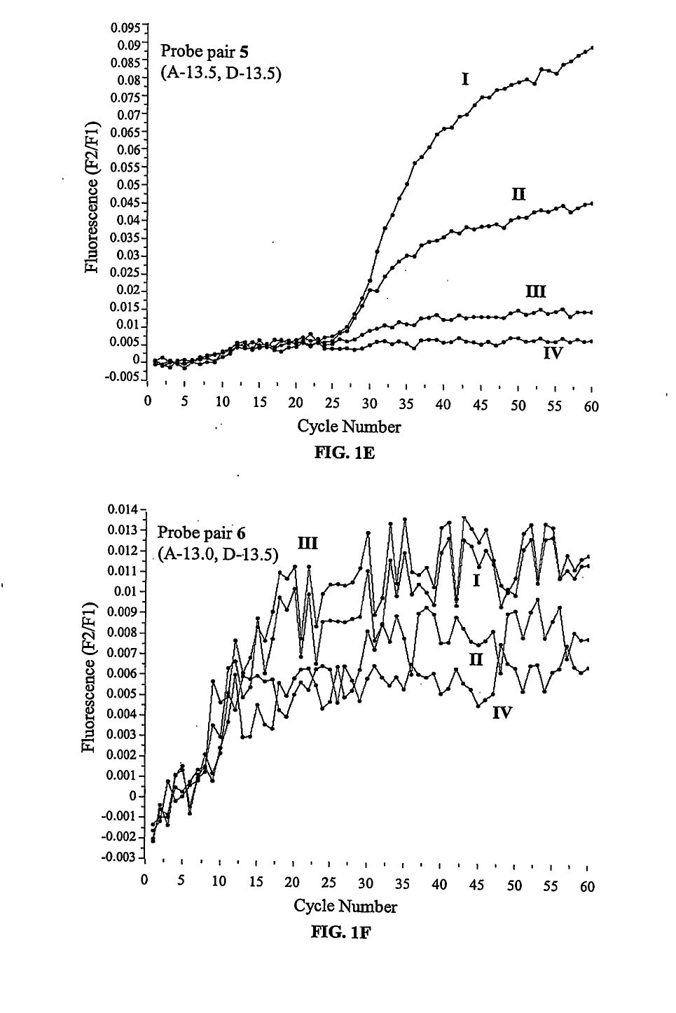

With reference to Example 4, the LightCycler™ analogous hybridization probe pairs 1 to 6 (Table 1) carrying fluorescein as the donor dye and LC Red 640 as the acceptor dye were used in separate real-time analytical experiments in the course of the PCR synthesis of an amplicon containing the SNP site G542X performed in the presence of the wild type, heterozygous type and mutant type template DNA, respectively. Each probe of these probe pairs that is derivatized with fluorescein is comprised of a sequence complementary to the sequence of the wild type template containing the site of the SNP G542X. The results are depicted graphically as relative fluorescence intensities versus cycle number in FIGS. 1A-F. As can be seen in FIG. 1A, which depicts the fluorescence intensity versus PCR cycle number for probe pair 1, which is comprised of DNA only, the discrimination of the wild type (I) from the mutant type (III) is acceptable, whereas it is barely distinguishable from the heterozygous type (II). The probe pairs 2 to 5, as listed in Table 1, are comprised of 3 to 5 monomeric LNA moieties in each probe, with one moiety positioned opposite to the SNP site. The results for these probe pairs, which are displayed in the FIGS. 1B to 1E demonstrate that the wild type (I), the heterozygous type (II) and the mutant type (III) are clearly differentiated. Additionally, the probes with a higher LNA content show improved discrimination. Thus, the 13-mer probes of probe pair 5 containing monomeric LNA moieties, as opposed to the 22- and 25-mer deoxynucleotides of probe pair 1, gave the most convincing results. Moreover, the 13-mers of the pair 6 comprising DNA only, employed for comparison purposes, did not yield any significant increase in fluorescence as shown in FIG. 1F, because the Tₘ values of their duplexes with the amplicon are too low.

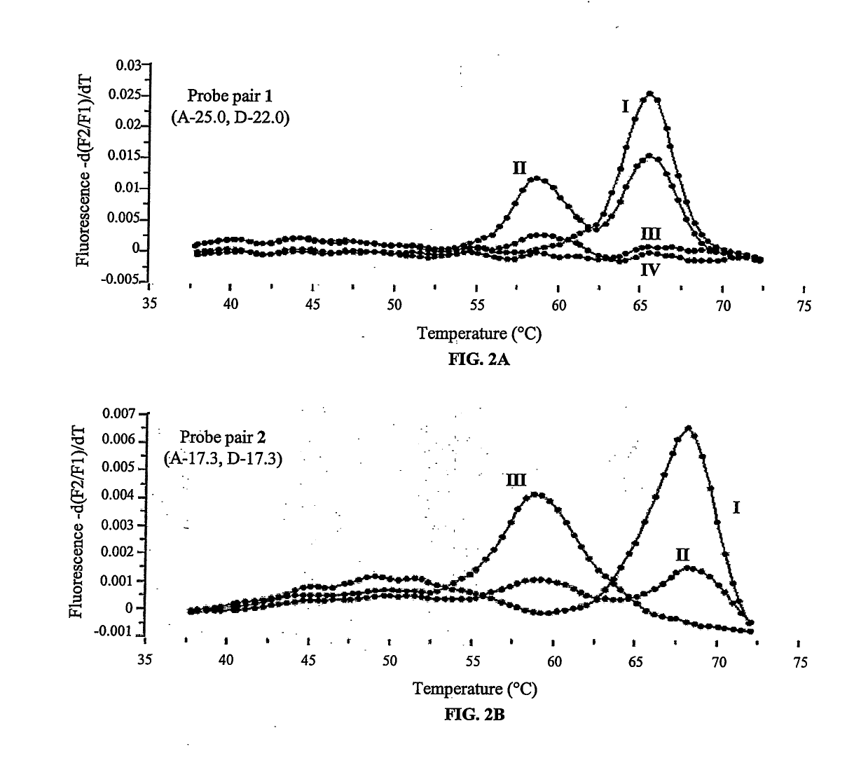

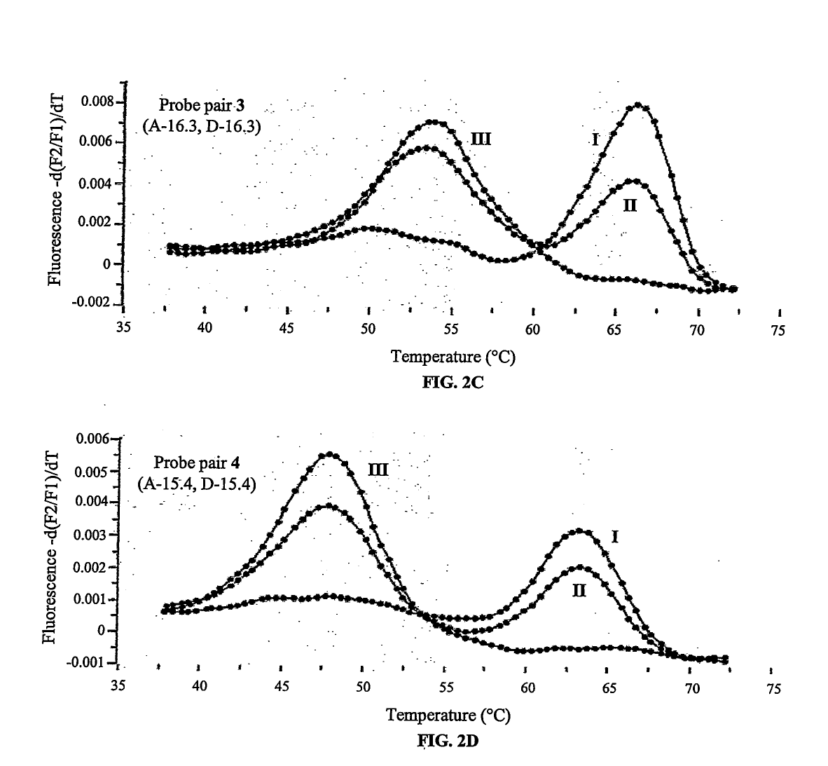

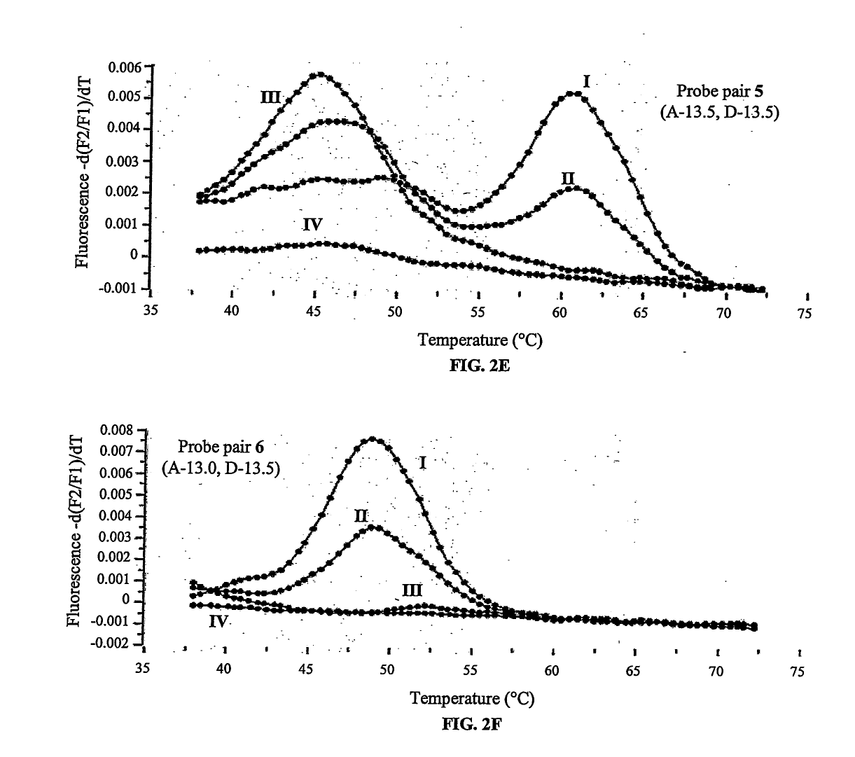

The subsequently measured time derivative melting curves for probe pairs 1 to 6, which are set forth in FIGS. 2A to 2F and described in Example 4, support these findings. Each probe/target duplex has a characteristic thermal stability that depends on such factors as length, G/C base pair content, sequence order, Watson-Crick pairing and LNA content. Base pair mismatches shift the stability of a duplex by varying amounts depending on the particular mismatch, the mismatch position, and neighboring base pairs. When a probe hybridizes over a sequence variant, a mismatch is formed and the duplex is destabilized. This is reflected by a shift in melting temperature (Tₘ) from the completely complementary duplex. The Tₘ value of a hybridizing probe is the temperature at which 50% of the probe has strand-separated from template sequence and can be estimated from the inflection point of the melting curve or the maximum of the corresponding time derivative curves, as those shown in FIGS. 2A-F.

FIG. 2A demonstrates that the Tₘ value for the duplex of wild type with the 22- and 25-mer deoxynucleotides of probe pair 1 (I) differs from that for the duplex of the mutant type (III) by 6° C. (ΔTₘ=6° C.). As displayed in FIGS. 2B to 2E and summarized in Table 2, the corresponding ΔTₘ values for the probe pairs 2 to 5 increase significantly as the LNA content of the probes rises. Thus, the probe pair 5 resulted in a ΔTₘ of 16° C., whereas the respective pair 6 without any LNA moiety, completely failed to provide a reasonable signal.

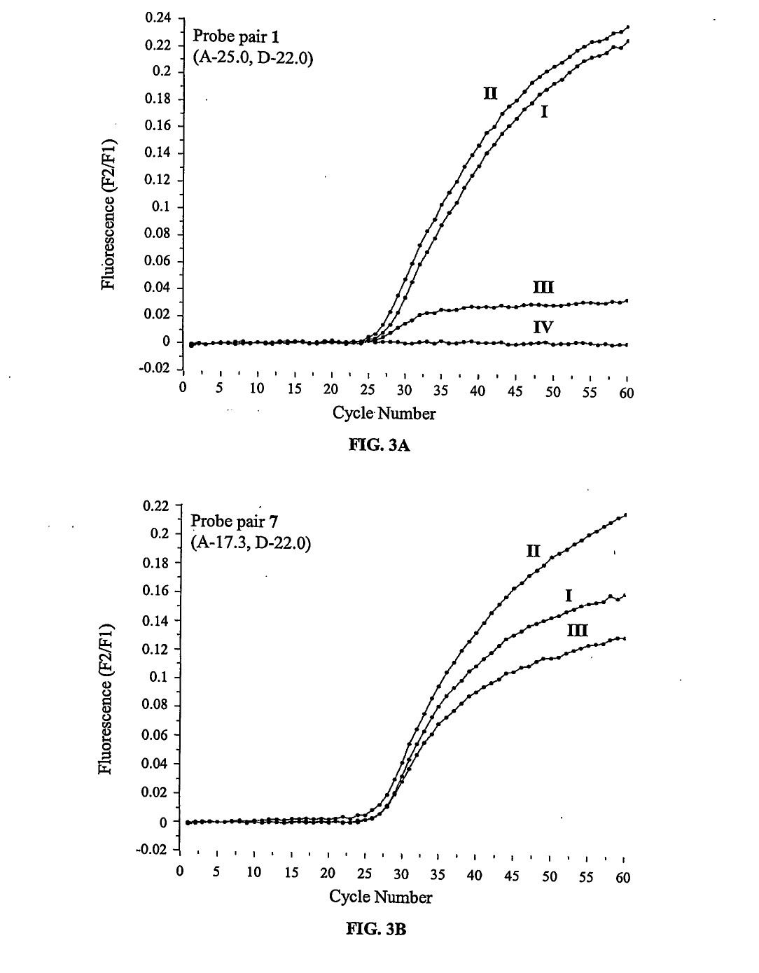

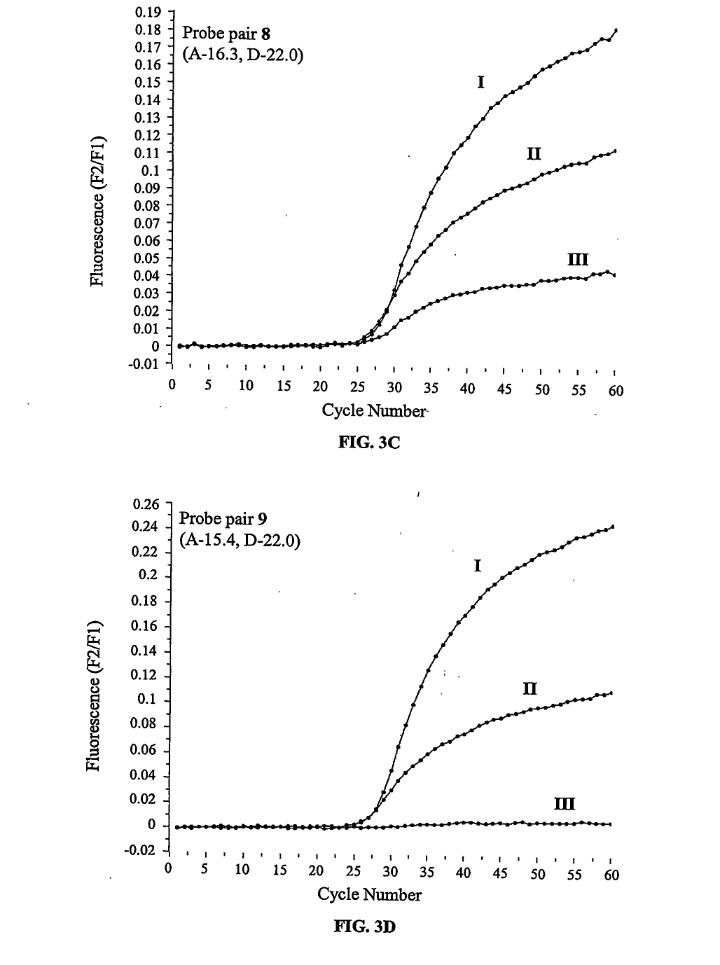

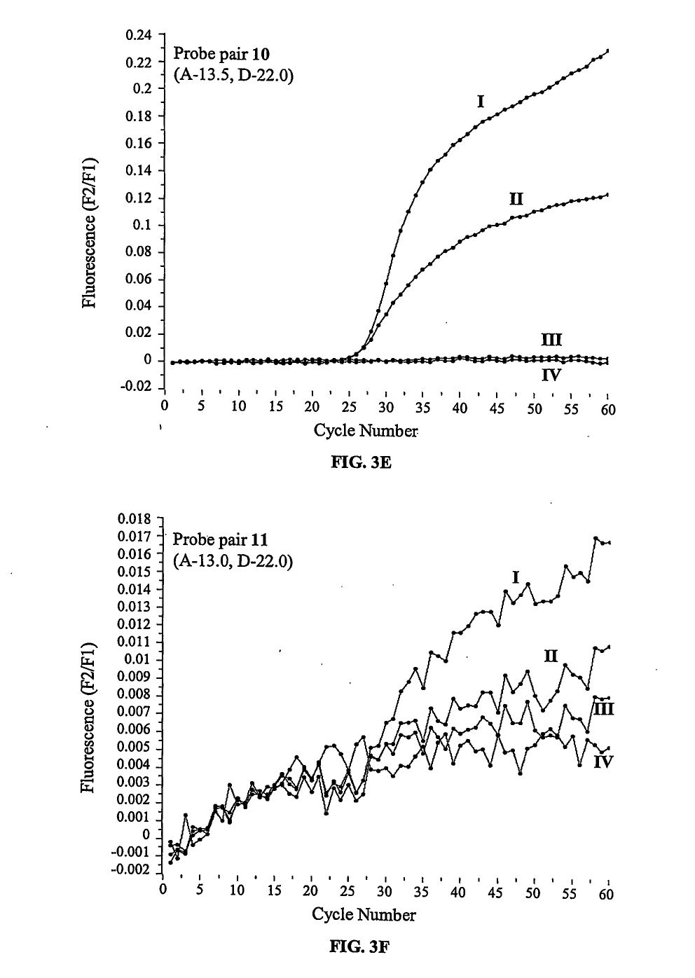

The same set of experiments was carried out with the probe pairs 7 to 10 as described in Example 5 and Table 3. Probe pairs 7 to 10 comprise the identical LC Red 640 derivatized probes as the aforementioned pairs 2 to 6, respectively, and the fluorescein derivatized deoxynucleotide probe D-22.0. FIGS. 3A to 3F display the fluorescence intensity versus PCR cycle number for these probes in comparison to the pure DNA probe pairs 1 and 11. The latter one (probe pair 11) having probes of the same length and sequence as probe pair 10 comprising the acceptor dye derivatized probe A-13.5, which is the shortest one with the highest LNA content. The results with regard to the discrimination of the wild and mutant types are quite similar and even slightly better than those for probe pairs 2 to 5, all of which contain LNA in each probe. In fact, the respective time derivative melting curves depicted in FIGS. 2A to 2F and the respective ΔTₘ values listed in Table 4, illustrate a further improved discrimination between wild and mutant types. Thus, a ΔTₘ of 19° C. was achieved with probe pair 10, as opposed to only 6° C. for the probe pair having a much longer LC Red 640 carrying probe, which, does not contain any LNA.

The use of the fluorogenic nucleic acid probes and pairs of nucleic acid probes of this invention in assays to detect and/or quantify nucleic acid analytes offers several advantages over the related prior art. The advantages are a result of the LNA mediated very high discrimination ability of the fluorescent labeled nucleic acid probes defined herein. Interestingly, as demonstrated herein, hybridization probe pairs with one probe including monomeric LNA moieties and a second probe without such moieties, may result in an even greater increase discrimination than a pair with LNA in both probes.

The probes and pair of probes as well as the attendant methods of the invention are useful in genotyping assays, in particular those for SNP detection. Furthermore, the compounds and methods of the invention are well suited to be employed for real-time assaying of PCR related investigations, such as for example allele specific PCR.

The following examples are provided for illustrative purposes only and are not intended to limit the scope of the invention.

### EXAMPLES

### Example 1

### General Procedure for the Preparation of the Labelled Oligo Probes

Oligonucleotide primers and probes were synthesized on a UFPS-24 synthesizer (Proligo LLC, Boulder, Colo., USA) using standard phosphoramidite chemistry, as known in the art. The 3′-fluorescein-labelled probes were synthesized using a fluorescein labelled CPG (Roche Diagnostics, Indianapolis, Ind., USA). The resulting labelled probes were purified by reverse phase high pressure liquid chromatography (HPLC) (Waters Symmetry Column, 5 μm, 3.9×150 mm, C18). The purities of these probes were determined by analytical reversed phase HPLC with monitoring at wavelengths of 260 nm and 495 nm.

The LC Red 640 labelled probes were synthesized using a phosphate-on CPG (Proligo LLC, Boulder, Colo., USA). Amino functionalization of the 5′-terminus was achieved by adding an amino modifying amidite with a C6 linker (Glen Research, Sterling, Va., USA) in the last synthetic cycle. After the deprotection and desalting steps the oligonucleotide was coupled via its amino group to the LC Red 640 dye carrying a NHS ester group by a manual labelling step according to the instructions provided by the supplier. The labelled oligonucleotides were purified first by precipitation to remove the excess Red 640, followed by reverse phase HPLC (Waters Symmetry Column, 5 μm, 3.9×150 mm, C18) of the resulting solution. The purity of the probes was then determined by analytical reversed phase HPLC at wavelengths of 260 nm and 625 nm.

### Example 2

### General Procedure for Performing Real-Time PCR Experiments with Hybridization Probe Pairs

The experiments were performed on a LightCycler™ thermal cycler (Roche Diagnostics, Indianapolis, Ind., USA). The PCR reactions were set up in a total volume of 25 μL with each tube containing standard PCR buffer (10×, 2.5 μL), MgCl₂ (4 mM), the deoxynucleotide triphosphates dATP, dGTP, dCTP and dTTP (200 μM each), the forward and reverse primers 5′-agg aag atg tgc ctt tca-3′ (SEQ ID NO:1) and 5′-aaa tgc ttg cta gac caa t-3′ (SEQ ID NO:2) (500 nM each), template DNA (10 ng), FastStart™ Taq DNA-Polymerase (1 unit, Roche Diagnostics, Indianapolis, Ind., USA), BSA (0.5 mg/mL), the probe derivatized with fluorescein (200 nM) and the probe derivatized with LC Red 640 (400 nM). The reactions were initiated at 95° C. for 7 minutes, followed by 60 cycles of denaturation at 95° C. for 10 seconds, annealing at 72° C. for 10 seconds and elongation at 72° C. for 15 seconds. The fluorescence intensities were recorded as a function of the cycle number in relation to the background fluorescence of a sample that was processed as specified above except that no template was added.

### Example 3

### General Procedure for Measuring Melting Curves of Hybridization Probe Pairs

Following real time PCR experiments as performed according to Example 2 the samples, still located in the LightCycler™ thermal cycler, were subjected to the following temperature profile: 95° C. for 30 seconds, 40° C. for 30 seconds and heating from 40 to 75° C. at a rate of 0.1° C. per second. The fluorescence intensities were recorded as a function of temperature in relation to the background fluorescence of a sample that was processed as specified above except that no template was added.

### Example 4

### Real-Time PCR and Subsequent Melting Experiments with Hybridization Probe Pairs 2 to 5 Comprising LNA Moieties in Both Probes

Probe pairs 2 to 5, as listed in Table 1 and prepared according to the procedure described in Example 1, were employed in real-time PCR experiments, which were performed pursuant to the general procedure described in Example 2. Separate experiments were conducted employing as template human DNA comprising either the wild type, the mutant type or the heterozygous type of the SNP G542X in the human cystic fibrosis (CF) gene CFTR. In all cases a 201 base pair amplicon corresponding to the sequence stretching from base pair 373 to base pair 573 of the CFTR gene was synthesized. The results are depicted graphically as relative fluorescence intensities versus cycle number in FIGS. 1B to 1E. For purposes of comparison, the results for the probe pairs 1 and 6, which contain deoxynucleotides only, are displayed in FIGS. 1A and 1F. The probes of probe pair 6 have the same base sequence and length as those of the probes of probe pair 5.

TABLE 1
Hybridization Probe Pairs Employed in Example 4

| Probe | Probe with acceptor | Probe | Probes with donor | Probe |
|---|---|---|---|---|
| Pair | dye LC Red 640* | ID | dye fluorescein* | ID |
| 1 | 5′-cca cct tct cca aga acta tat tgt-3′ | A-25.0 | 5′-cgt tga cct cca ctc agt gtg a-3′ | D-22.0 |
| | (SEQ ID NO:3) | | (SEQ ID NO:4) | |
| 2 | 5′-cca cCt tct cCa agA ac-3′ | A-17.3 | 5′-a ccT cca Ctc agT gtg a-3′ | D-17.3 |
| | (SEQ ID NO:5) | | (SEQ ID NO:6) | |
| 3 | 5′-cca cct tct CCA aga ac-3′ | A-16.3 | 5′-cct cca CTC agt gtg a-3′ | D-16.3 |
| | (SEQ ID NO:7) | | (SEQ ID NO:8) | |
| 4 | 5′-ct tcT CCa agA act a-3′ | A-15.4 | 5′-Ca cTc aGt gtG att cc-3′ | D-15.4 |
| | (SEQ ID NO:9) | | (SEQ ID NO:10) | |
| 5 | 5′-CT tct CCa aGa ac-3′ | A-13.5 | 5′-Ctc agT Gtg Att Cc-3′ | D-13.5 |
| | (SEQ ID NO:11) | | (SEQ ID NO:12) | |
| 6 | 5′-ct tct cca aga ac-3′ | A-13.0 | 5′-ctc agt gtg att cc-3′ | D-13.0 |
| | (SEQ ID NO:13) | | (SEQ ID NO:14) | |

*Capital letters represent monomeric LNA moieties. Bold letters represent the nucleotides of the probes derivatized with a donor dye (LC Red 640) that are positioned opposite to the SNP site of the template following the hybridization event.

Following the PCR experiments, the melting curves of the amplicons in the presence of each of the hybridization probe pairs 1 to 6 were measured according to the general procedure described in Example 3. The results are depicted graphically as relative fluorescence intensities versus temperature in FIGS. 2A to 2F. For each probe pair the ΔTₘ value, as listed in Table 2, was determined by subtracting the melting point measured with the wild type template from that measured with the mutant type template.

TABLE 2
ΔTₘ values determined according to Example 4

| Probe Pair | Probe IDs | ΔTₘ* |
|---|---|---|
| 1 | A-25.0, D-22.0 | 6° C. |
| 2 | A-17.3, D-17.3 | 9° C. |
| 3 | A-16.3, D-16.3 | 12° C. |
| 4 | A-15.4, D-15.4 | 15° C. |
| 5 | A-13.5, D-13.5 | 16° C. |
| 6 | A-13.0, D-13.0 | 0° C. |

*The ΔTₘ values represent the difference between melting temperatures of the duplexes formed by probe pairs 1 to 6, respectively, with the amplicon derived from the human template DNA comprising a wild type SNP G542X in the CFTR gene, and those of the corresponding duplexes involving the amplicon containing the mutant type SNP.

### Example 5

### Real-Time PCR and Subsequent Melting Experiments with Hybridization Probe Pairs 7 to 10 Comprising LNA Moieties Only in the Probes Carrying the Donor Dye

Probe pairs 7 to 10, as listed in Table 3 and prepared according to the procedure described in Example 1, were employed in real-time PCR experiments that were performed pursuant to the general procedure of Example 2. Separate experiments were conducted employing as template human DNA comprising either the wild type, the mutant type or the heterozygous type of the SNP G542X in the human cystic fibrosis (CF) gene CFTR. In all cases a 201 base pair amplicon corresponding to the sequence stretching from base pair 373 to base pair 573 of the CFTR gene was synthesized. The results are depicted graphically as relative fluorescence intensities versus cycle number for probe pairs 7 to 10 in FIGS. 3B to 3E. For purposes of comparison, the results for probe pairs 1 and 11 that exclusively contain deoxynucleotides are displayed in FIGS. 3A and 3F. The LC Red 640 derivatized probe of probe pair 11 has the same base sequence and length as the corresponding probe of pair 10.

TABLE 3
Hybridization probe pairs employed in Example 5

| Probe | Probe with acceptor | Probe | Probes with donor | Probe |
|---|---|---|---|---|
| Pair | dye LC Red 640* | ID | dye fluorescein* | ID |
| 1 | 5′-cca cct tct cca aga acta tat tgt-3′ | A-25.0 | 5′-cgt tga cct cca ctc agt gtg a-3′ | D-22.0 |
| | (SEQ ID NO:3) | | (SEQ ID NO:4) | |
| 7 | 5′-cca cCt tct cCa agA ac-3′ | A-17.3 | 5′-cgt tga cct cca ctc agt gtg a-3′ | D-22.0 |
| | (SEQ ID NO:5) | | (SEQ ID NO:4) | |
| 8 | 5′-cca cct tct CCA aga ac-3′ | A-16.3 | 5′-cgt tga cct cca ctc agt gtg a-3′ | D-22.0 |
| | (SEQ ID NO:7) | | (SEQ ID NO:4) | |
| 9 | 5′-ct tcT CCa agA act a-3′ | A-15.4 | 5′-cgt tga cct cca ctc agt gtg a-3′ | D-22.0 |
| | (SEQ ID NO:9) | | (SEQ ID NO:4) | |
| 10 | 5′-CT tct CCa aGa ac-3′ | A-13.5 | 5′-cgt tga cct cca ctc agt gtg a-3′ | D-22.0 |
| | (SEQ ID NO:11) | | (SEQ ID NO:4) | |
| 11 | 5′-ct tct cca aga ac-3′ | A-13.0 | 5′-cgt tga cct cca ctc agt gtg a-3′ | D-22.0 |
| | (SEQ ID NO:15) | | (SEQ ID NO:4) | |

*Capital letters represent monomeric LNA moieties. Bold letters represent the nucleotides of the probes derivatized with a donor dye (LC Red 640) that are positioned opposite to the SNP site of the template following the hybridization event.

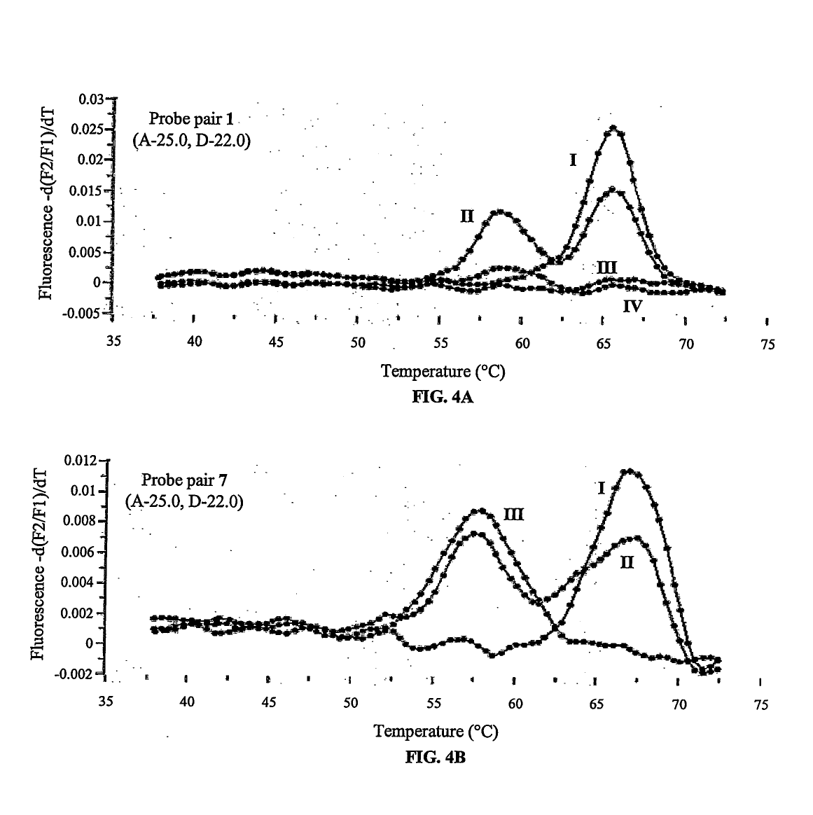

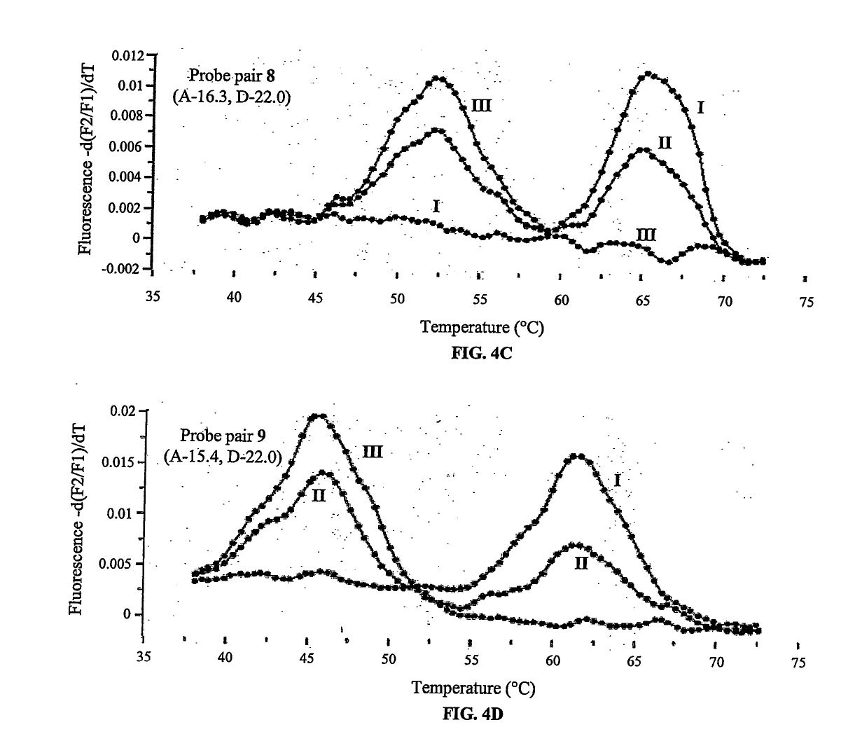

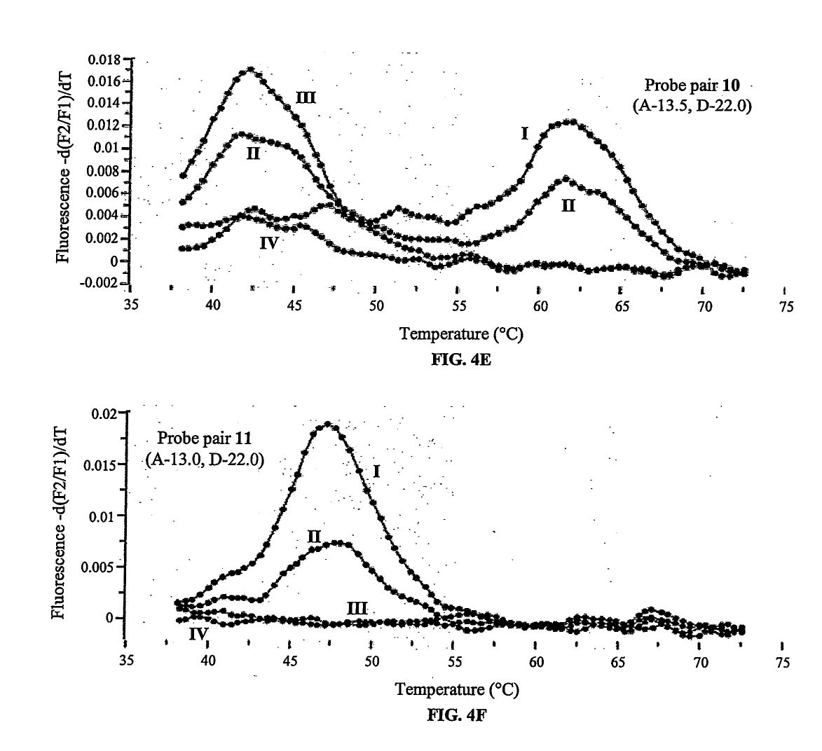

Following the PCR experiments the melting curves of the amplicons in the presence of each of the hybridization probe pairs 1 and 7 to 11 were measured according to the general procedure of Example 3. The results are depicted as relative fluorescence intensities versus temperature in FIGS. 4A to 4F. For each probe pair the ΔTₘ value, as listed in Table 4, was determined by subtracting the melting point measured with the wild type template from those measured with the mutant type template.

TABLE 4
ΔTₘ values determined according to Example 5

| Probe Pair | Probe IDs | ΔTₘ* |
|---|---|---|
| 1 | A-25.0, D-22.0 | 6° C. |
| 7 | A-17.3, D-22.0 | 11° C. |
| 8 | A-16.3, D-22.0 | 13° C. |
| 9 | A-15.4, D-22.0 | 16° C. |
| 10 | A-13.5, D-22.0 | 19° C. |
| 11 | A-13.0, D-22.0 | 0° C. |

The ΔTₘ values represent the difference between melting temperatures of the duplexes formed by the probe pairs 1 and 7 to 11, respectively, with the amplicon derived from the human template DNA comprising a wild type SNP G542X in the CFTR gene, and those of the corresponding duplexes involving the amplicon containing the mutant type SNP.

1. A nucleic acid probe comprised of an n-meric nucleic acid comprising any number of 1 to n monomeric linked nucleic acid (LNA) moieties that may be situated in any position(s) of the nucleic acid sequence, wherein said nucleic acid probe is derivatized with at least one dye and wherein n is an integer selected from 1-200.

2. The nucleic acid probe of claim 1, wherein said probe is complementary or largely complementary to a section of a nucleic acid analyte comprising a single nucleotide polymorphism (SNP) site, wherein one monomeric LNA moiety is positioned opposite to the SNP site subsequent to the hybridization of the probe with the analyte.

3. The nucleic acid probe of claim 2 wherein said monomeric LNA moiety is complementary to the opposing SNP site of the nucleic acid analyte.

4. The nucleic acid probe of claim 2 wherein said monomeric LNA moiety is not complementary to the opposing SNP site of the nucleic acid analyte.

5. The nucleic acid probe of claim 1, wherein said probe is derivatized with two or more non-identical covalently attached dyes, wherein at least one of said dyes is a fluorescent dye.

6. The nucleic acid probe of claim 5, wherein said probe is comprised of a fluorescent dye and a non-fluorescent quencher dye.

7. The nucleic acid probe of claim 5, wherein said probe is comprised of two different fluorescent dyes, wherein said fluorescent dyes are able to jointly constitute the donor dye and the acceptor dye, respectively, of a FRET system.

8. A pair of nucleic acid probes comprised of either two nucleic acid probes of claim 1 or one nucleic acid probe of claim 1 and another nucleic acid probe that is derivatized with at least one dye, wherein both probes comprise nucleic acids having nucleotide sequences differing from each other, that are complementary or largely complementary to adjacent segments of the target sequence of the nucleic acid analyte, wherein the two probes are collectively derivatized with two or more non-identical covalently attached dyes, wherein at least one dye is fluorescent, and wherein each probe comprises at least one of said dyes.

9. The pair of nucleic acid probes according to claim 8, comprising a fluorescent dye and a non-fluorescent quencher dye.

10. The pair of nucleic acid probes according to claim 8, comprising two fluorescent dyes, wherein said fluorescent dyes are able to jointly constitute the donor dye and the acceptor dye, respectively, of a FRET system.

11. A method for detection or quantification of a nucleic acid analyte comprising the steps of:
a.) providing a nucleic acid probe, wherein said nucleic acid probe is comprised of at least one monomeric LNA moiety and with two or more non-identical covalently attached dyes, wherein at least one dye is fluorescent;
b.) contacting said nucleic acid probe with the nucleic acid analyte so as to allow for the hybridization of the nucleic acid probe with the nucleic acid analyte; and
c.) measuring the change in the fluorescence of the nucleic acid probe that is related to the hybridization of the nucleic acid probe with the nucleic acid analyte.

12. The method of claim 11 wherein the nucleic acid probe comprises a fluorescent dye and a non-fluorescent quencher dye.

13. The method of claim 11 wherein the nucleic acid probe comprises a donor dye and an acceptor dye, respectively, which are able to jointly constitute a FRET system.

14. The method of claim 11 carried out as a homogeneous assay to detect or quantify a nucleic acid analyte in a sample.

15. The method of claim 11 wherein said change in the fluorescence occurs upon the hybridization of the nucleic acid probe with the nucleic acid analyte.

16. The method of claim 11 wherein said change in the fluorescence occurs upon the hydrolysis of the nucleic acid probe as hybridized with the nucleic acid analyte.

17. The method of claim 14 wherein the homogeneous assay is a polymerase chain reaction.

18. The method of claim 17 wherein said nucleic acid probe functions as a hybridization probe in a polymerase chain reaction, providing for a real-time detection or quantification of the amplification product.

19. The method of claim 11 wherein the nucleic acid probe is adapted for use as Molecular Beacon.

20. The method of claim 16 wherein the probe is hydrolyzed during the DNA synthesis steps of the temperature cycles of the polymerase chain reaction.

21. The method of claim 20 wherein the nucleic acid probe is adapted for the use as a Taqman probe.

22. The method of claim 11 conducted in a multiplexed format.

23. The method of claim 11 for analyzing a SNP site of a nucleic acid analyte, wherein said nucleic acid probe comprises a monomeric LNA moiety that is positioned opposite to the SNP site subsequent to the hybridization of the probe with the analyte.

24. A method for detection or quantification of a nucleic acid analyte comprising the steps of:
a.) providing a pair of nucleic acid probes, wherein said probes differ in their nucleic acid sequences, and wherein said probes collectively include at least one monomeric LNA moiety and are collectively derivatized with two or more non-identical covalently attached dyes, wherein at least one dye is fluorescent, and wherein the each probe comprises at least one of said dyes.
b.) contacting said pair of nucleic acid probes with the nucleic acid analyte so as to allow for the hybridization of the pair of nucleic acid probes with the nucleic acid analyte in such a way that both probes are hybridized to adjacent segments of the target sequence of the nucleic acid analyte; and
c.) measuring the change in the fluorescence of the pair of nucleic acid probes that is related to the hybridization of the nucleic acid probe with the nucleic acid analyte.

25. The method of claim 24 wherein the pair of nucleic acid probes comprises a fluorescent dye and a non-fluorescent quencher dye.

26. The method of claim 24 wherein the pair of nucleic acid probes comprises a donor dye and an acceptor dye, respectively, which are able to jointly constitute a FRET system.

27. The method of claim 26 wherein upon said hybridization of the pair of nucleic acid probes with the nucleic acid analyte the donor and the acceptor dyes are within 25 nucleotides of one another.

28. The method of claim 27 wherein donor dye is fluorescein and the acceptor dye is Cy5 or Cy5.5.

29. The method of claim 27 wherein donor dye is fluorescein and the acceptor dye is LC Red 640 or LC Red 705.

30. The method of claim 24 carried out as a homogeneous assay to detect or quantify a nucleic acid analyte in a sample.

31. The method of claim 24 wherein said change in the fluorescence occurs upon the hybridization of both probes of the pair of nucleic acid probes with the nucleic acid analyte.

32. The method of claim 24 wherein said change in the fluorescence occurs upon the removal of at least one of the probes as hybridized with the nucleic acid analyte.

33. The method of claim 30 wherein the homogeneous assay is a polymerase chain reaction.

34. The method of claim 32 wherein at least one of the probes is removed during the DNA synthesis steps of the temperature cycles of the polymerase chain reaction.

35. The method of claim 33 wherein said pair of nucleic acid probes functions as a pair of hybridization probes in a polymerase chain reaction, providing for a real-time detection or quantification of the amplification product.

36. The method of claim 35 wherein the pair of nucleic acid probes is adapted for the use as LightCycler probes.

37. The method of claim 24 conducted in a multiplexed format.

38. The method of claim 24 for analyzing a SNP site of a nucleic acid analyte, wherein said pair of nucleic acid probes comprises a monomeric LNA moiety that is positioned opposite to the SNP site subsequent to the hybridization of the probes with the analyte.
